(19) United States
(12) Patent Application Publication
(10) Pub. No.: US 2016/0130489 A1
(43) Pub. Date: May 12, 2016

(54) DUST SUPPRESSANT

(71) Applicants: NOVOZYMES A/S, Bagsvaerd (DK); EARTH ALIVE CLEAN TECHNOLOGIES INC., Montreal, Québec (CA)

(72) Inventor: David Gilmour, Westmount, Quebec (CA)

(21) Appl. No.: 14/897,927

(22) PCT Filed: Jun. 12, 2014
(86) PCT No.: PCT/EP2014/062265

§ 371 Date: Dec. 11, 2015

Related U.S. Application Data
(60) Provisional application No. 61/833,969, filed on Jun. 12, 2013.

(51) Int. Cl. C09K 3/22; E01C 21/00; E01C 7/36; C12N 1/20

(52) U.S. Cl. CPC C09K 3/22; C12N 1/20; E01C 21/00; E01C 7/36

(57) ABSTRACT

A dust suppressant composition and a method for suppressing dust particularly on roads, the dust suppressant composition comprising a carrier and one or more dust suppressing microbes.

### FIELD OF THE INVENTION

The present invention relates to a dust suppressant and more particularly, relates to dust suppressant compositions and a method for suppressing dust.

### BACKGROUND OF THE INVENTION

The use of dust suppressants is well known in the art and typically, such dust suppressants are utilized on roads to suppress dust on an unpaved surface.

There are several main areas of concern which provoke the need for dust abatement. This includes environmental considerations as the dust is capable of contamination of waterways. The dust is also a problem as far as contamination of soil and plants. In areas of glaciers, the dust will affect the rate of melting of the glaciers.

Dust abatement is also necessary for health and safety reasons. Thus, the inhalation of dust is undesirable for contamination of the lungs and subsequent health issues. In some instances, it can cause poor visibility for traffic and lead to unsafe situations.

It is also desirable to suppress the dust which can cause wear and tear on mechanical equipment as well as requiring more frequent repair.

In many situations, it is considered desirable and even essential to suppress dust particularly when the dust is created by vehicular movement. The dust can cause many problems including visibility and pollution. Thus, the dust can inhibit visibility for subsequent vehicles. Furthermore, although most vehicles have an air filter, when they are used in an area of persistent dust, the filters rapidly become clogged and there is greater wear on the engine. This usually requires very frequent filter changes along with oil changes.

Originally, many locations utilized used motor oil as a dust suppressant. In today's environment, this is no longer ecologically acceptable. Many different chemical products have been proposed to control dust; while some are reasonably effective, each usually has drawbacks associated therewith.

A further problem which is known in the art and which is very severe is the dust raised by trucks or other heavy machinery on dirt roads. This problem is particularly prevalent at some mining sites where trucks are continually utilizing the road. Frequently, these sites are ecologically important and a surplus of dust will pollute the water and hurt many small farming communities. Furthermore, at higher altitudes, the dust will accumulate on the snow and cause quicker melting.

In coal-mining applications, mechanical and chemical methods for dust control are known. For example, dust-collection equipment is used in mining operations. Also, water is commonly used to prevent dust particles from becoming airborne. Additionally, aqueous solutions containing surfactants may be used for dust control (see e.g., U.S. Pat. No. 3,690,727 and U.S. Pat. No. 4,136,050).

Aqueous foam compositions have also been used to suppress dust (see e.g., U.S. Pat. No. 3,954,662, U.S. Pat. No. 4,000,992, and U.S. Pat. No. 4,400,220). U.S. Pat. No. 4,316,811 discloses the use of an aqueous solution of polyethylene oxide for dust control. U.S. Pat. No. 4,169,170 discloses the use of an aqueous composition comprising an asphalt emulsion or a black liquor lignin product and a water-soluble methoxylated alkylphenol or sulphosuccinate wetting agent to form a crust layer, which provides protection against the loss of coal due to wind or the action of a coal-transportation device.

Emulsions have also been used to suppress dust. U.S. Pat. No. 4,650,598 discloses a dust suppressing emulsion comprising (a) 20-99.5%, by weight, water and (b) the balance a composition comprising at least one methacrylate polymer, at least one hydrophobic liquid, and at least one emulsifying surfactant. U.S. Pat. N o. 4,650,598 further discloses methods for suppressing dust with the aforementioned emulsion.

### SUMMARY OF THE INVENTION

Notwithstanding the above mentioned dust suppressing methods and compositions, it is an object of the present invention to provide a dust suppressant composition which is effective in controlling dust while also being ecologically acceptable (e.g., having positive/beneficial impacts on the environment such as bioremediations and/or soil regeneration, etc.).

In one embodiment, the dust suppressant composition comprises one or more microbes capable of suppressing (e.g., controlling, inhibiting, reducing) dust.

According to the present invention, one may provide an improved ecologically accepted dust suppressant composition, which has a liquid portion having a tackiness sufficient to bind dirt particles together, the improvement comprising adding hydrocarbon degrading microbes to the composition. In an embodiment, the improvement further comprises adding one or more microbes capable of suppressing dust. In still yet another embodiment, the one or more hydrocarbon degrading microbes are capable of suppressing dust. In still yet another embodiment, the one or more hydrocarbon degrading microbes are capable of emitting natural polymers to further bind said dirt particles together.

In a preferred aspect of the present invention, there is provided a method for reducing dust comprising the step of applying to a substrate a mixture of a liquid glycerine, water, a natural polymer, and one or more microbes capable of suppressing dust.

### BRIEF DESCRIPTION OF THE DRAWINGS

FIG. 1 is graph showing average dust concentrations calculated for each road based on mobile monitoring (Example 1);

FIG. 2 shows maximum dust concentrations measured for each road based on mobile monitoring (Example 1);

FIG. 3 shows average dust concentrations calculated for each road based on static monitoring (Example 1); and

FIG. 4 shows maximum dust concentrations measured for each road based on static monitoring (Example 1).

### DETAILED DESCRIPTION OF THE EMBODIMENTS

The disclosed embodiments relate to compositions and methods for suppressing dust.

### Definitions:

As used herein, the singular forms “a”, “an” and “the” are intended to include the plural forms as well, unless the context clearly indicates otherwise.

As used herein, the term “beneficial microorganism(s)” or “beneficial microbe(s)”, etc. is intended to mean any microorganism (e.g., bacteria, fungus, etc., or combination thereof), regardless of whether the microorganism is in a vegetative state or spore form, that is capable of causing or providing a beneficial and/or useful effect (e.g., hydrocarbon degradation, dust suppression, polymer production, etc.) when applied to a substrate.

As used herein, the term “beneficial ingredient(s)” is intended to mean any agent or combination of agents capable of causing or providing a beneficial and/or useful effect in dust suppression.

As used herein, the terms “dust suppression”, “dust suppressing”, etc. is intended to mean the prevention of dust, control of dust, the inhibition of dust, the reduction of dust, or the elimination of dust to the extent to which fine particulates become airborne or suspended in air. By “dust” is meant any particulate solid material that is susceptible to suspension in air or other atmospheric environment. Accordingly, the term “dust” is intended to include particles having an average diameter of up to 1 cm, preferably up to 1 mm, (though typically only up to about 600 or about 300 micron) and down into the fume range (e.g., typically as low as 0.001 micrometers). Dust particles include particles of organic matter, such as spices or textile dust, and mineral based particles, such as sand; and combinations thereof. In a preferred embodiment, the soil particles include silica (silicon dioxide); more preferably, the soil particles include silica as the major component; and most preferably, the soil particles are essentially made of silica.

As used herein, the term “isomer(s)” is intended to include all stereoisomers of the compounds and/or molecules referred to herein (non-limiting examples include, proteins, metabolites (such as primary metabolites, secondary metabolites, etc.), polymers, polyols (such as glycerine) lipids, fats, oils, triglycerides, enzymes, etc.), including enantiomers, diastereomers, as well as all conformers, rotamers, and tautomers, unless otherwise indicated. The compounds and/or molecules disclosed herein include all enantiomers in either substantially pure levorotatory or dextrorotatory form, or in a racemic mixture, or in any ratio of enantiomers. Where embodiments disclose a (D)-enantiomer, that embodiment also includes the (L)-enantiomer; where embodiments disclose a (L)-enantiomer, that embodiment also includes the (D)-enantiomer. Where embodiments disclose a (+)-enantiomer, that embodiment also includes the (−)-enantiomer; where embodiments disclose a (−)-enantiomer, that embodiment also includes the (+)-enantiomer. Where embodiments disclose a (S)-enantiomer, that embodiment also includes the (R)-enantiomer; where embodiments disclose a (R)-enantiomer, that embodiment also includes the (S)-enantiomer. Embodiments are intended to include any diastereomers of the compounds and/or molecules referred to herein in diastereomerically pure form and in the form of mixtures in all ratios. Unless stereochemistry is explicitly indicated in a chemical structure or chemical name, the chemical structure or chemical name is intended to embrace all possible stereoisomers, conformers, rotamers, and tautomers of compounds and/or molecules depicted.

As used herein, the terms “effective amount”, “effective concentration”, or “effective dosage” is intended to mean the amount, concentration, or dosage of the one or more microbes sufficient to suppress dust. The actual effective dosage in absolute value depends on factors including, but not limited to, the type of dust to be treated (such as particulate solid material, soil, stone or graveled path, clayed earth or sand), the humidity of the environment, synergistic or antagonistic interactions between the other active or inert ingredients which may enhance or reduce the dust suppressing effects of the one or more microbes, and the stability of the one or more microbes in compositions alone or in combination with one or more dust suppression treatments. The “effective amount”, “effective concentration”, or “effective dosage” of the one or more microbes may be determined by one skilled in the art, e.g., by a routine dose response experiment.

As used herein, the term “carrier” is intended to refer to any material which can be used to deliver the actives (e.g., microorganisms described herein, etc.) to a substrate in need of dust suppression.

As used herein, the terms “spore”, “microbial spore”, “bacterial spore”, etc., have their normal meaning which is well known and understood by those of skill in the art. As used herein, the terms “spore” and “microbial spore” refer to a microorganism in its dormant, protected state.

As used herein the expression “dust suppressing microbes” is meant to refer to microbes that have the ability to prevent dust to get airborne, that is microbes generally forming biofilm. As used herein the term “microbes” is intended to refer to microorganisms, preferably bacteria and fungi, and more preferably bacteria or bacterial spores.

### Compositions

The compositions disclosed comprise a carrier, one or more beneficial microorganisms as described herein. In certain embodiments, the composition may be in the form of a liquid, a gel, a slurry, a solid, or a powder (e.g., a wettable powder or a dry powder).

### Carriers

The carriers described herein will allow the microorganism(s) to remain efficacious (e.g., capable of suppressing dust, degrading hydrocarbons, etc.) and viable once formulated. Non-limiting examples of carriers described herein include liquids, slurries, or solids (including wettable powders or dry powders).

In an embodiment, the carrier is a slurry. In an embodiment, the slurry may comprise a sticking agent, a liquid, or a combination thereof. It is envisioned that the sticking agent can be any agent capable of sticking the one or more microorganisms described herein (e.g., one or more microorganisms capable of suppressing dust, one or more microorganisms capable of hydrocarbon degradation, etc.) to a substrate of interest (e.g., a soil, road, surface, etc.). Non-limiting examples of sticking agents include alginate, mineral oil, syrup, gum arabic, honey, methyl cellulose, milk, wallpaper paste, and combinations thereof. Non-limiting examples of liquids appropriate for a slurry include water and solutions, (e.g., aqueous solutions and non-aqueous solutions). An appropriate aqueous solution for a slurry may include sugar water. In a particular embodiment, an aqueous solution of water and glycerine is added to the slurry.

In another embodiment, the carrier is a solid. In a particular embodiment the solid is a powder. In one embodiment the powder is a wettable powder. In another embodiment, the powder is a dry powder. In another embodiment, the solid is a granule. Non-limiting examples of solids useful as carriers for the compositions disclosed herein include peat, wheat, wheat chaff, ground wheat straw, bran, vermiculite, cellulose, starch, soil (pasteurized or unpasteurized), gypsum, talc, clays (e.g., kaolin, bentonite, montmorillonite), and silica gels.

In a particular embodiment, the carrier is a liquid carrier. If a liquid carrier is used, the liquid carrier may further include growth media to culture one or more microbial strains used in the compositions described. Non-limiting examples of suitable growth media for microbial strains include YEM media, mannitol yeast extract, glycerol yeast extract, Czapek-Dox medium, potato dextrose broth, or any media known to those skilled in the art to be compatible with, and/or provide growth nutrients to the microbial strains which may be included to the compositions described herein. Non-limiting examples of liquids useful as carriers for the compositions disclosed herein include water, an aqueous solution, or a non-aqueous solution. In another embodiment the carrier is an organic solvent. In another embodiment, the carrier is a non-aqueous solution. In a particular embodiment the carrier is water. In another embodiment the carrier is an aqueous solution. In a further embodiment, the carrier is an aqueous solution comprising water and sugar (i.e., sugar water). In a particular embodiment the carrier is glycerine (e.g., liquid glycerine). In an even more specific embodiment, the glycerine is bio-based, composed of carbon and will biodegrade over a period of time.

In a particular embodiment, the carrier is an aqueous solution comprising water and glycerine. In a more particular embodiment, water is added to the liquid glycerine in approximately equal amounts. The formulation (e.g., the liquid carrier) and ingredients will vary greatly depending on soil type and road composition. Each road bed is different. Generally, the ratio of liquid glycerine can comprise between 10% and 60% by weight of the composition.

In still a more particular embodiment, the carrier is an aqueous solution comprising water and glycine and may further comprise one or more oils (e.g., agricultural based oils such as soy and vegetable oils, vegetable based oils, vegetable based oil emulsions, sulfonated oils, petroleum oils, parrafinic oils, etc.), one or more sugars (e.g., sugar water, glucose, fructose, high fructose syrups, corn syrup, molasses, galactose, sucrose, maltose, lactose, monosaccharides, disaccharides, polysaccharides, etc.), and one or more sugar alcohols (e.g., a polyol, glycol, glycerol, erythritol, theritol, arabitol, xylitol, ribitol, mannitol, sorbitol, galactitol, fucitol, iditol, inositol, volemitol, isomalt, maltitol, lactitol, malotriitol, malotetraitol, polyglycitol, etc.)

### Microorganisms

The compositions disclosed herein comprise one or more microorganisms. The microbes have been found to enhance the performance of the composition.

In an embodiment, the one or more microorganisms are one or more fungi. In another embodiment, the one or more microorganisms are one or more bacteria. In another embodiment, the one or more microorganisms are one or more bacteria capable of suppressing dust. In another embodiment, the one or more microorganisms are capable of degrading hydrocarbons. In still another embodiment, the one or more microorganisms are capable of suppressing dust and degrading hydrocarbons.

In a more particular embodiment, the one or more bacteria capable of suppressing dust are spore forming bacterial strains. In still a more particular embodiment, the one or more bacteria capable of degrading hydrocarbons are spore forming bacterial strains. In still another particular embodiment, the one or more microorganisms are capable of suppressing dust and degrading hydrocarbons are spore forming bacterial strains. Methods for producing stabilized microorganisms, and bacteria specifically, are known in the art. See Donnellan, J. E., Nags, E. H., and Levinson, H. S. (1964). “Chemically defined, synthetic media for sporulation and for germination and growth of Bacillus subtilis.” Journal of Bacteriology 87(2):332-336; and Chen, Z., Li, Q., Liu, H. Yu, N., Xie, T., Yang, M., Shen, P., Chen, X. (2010). “Greater enhancement of Bacillus subtilis spore yields in submerged cultures by optimization of medium composition through statistical experimental designs.” Appl. Microbiol. Biotechnol. 85:1353-1360.

Non-limiting examples of spore forming bacterial strains include strains from the genera Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and/ or Vulcanobacillus.

In a particular embodiment, the one or more spore forming bacteria is a bacteria selected from the genera consisting of Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridfisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Omithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof.

In a particular embodiment, the one or more microbes degrade hydrocarbons. Generally, the microbial content will attack and degrade one or more hydrocarbons, such as, phenol, benzene, toluene, other aromatic hydrocarbons with hydroxylated, nitrogenated groups, octane, ethane, and other short-chained alkyl hydrocarbons; salicylic acid, biphenyl, xylol, phenoxy alcohols, mineral oils, lubricating oils, kerosene, surfactants, gasoline, pentachlorophenol, intermediate length alkyl hydrocarbons and alcohols, fatty acids, benzolic acid and citrus oils;

complex dyes, lignins, starchy complexes, carbohydrate by-product waste, wood pulp waste, structural board and pressboard waste, distillery waste, wood preservative waste, creosols, creosote, naphthalene, ethylene glycol, and heterogeneous aromatic hydrocarbon waste, protein complex wastes, oleaginous waxes or fats containing wastes, wastes with fats and oils and dissolved aromatics, hydrocarbons linked with aminos, glycerol esters; treating fuel oils, intermediate levels of moderate molecular weight hydrocarbon contamination in soil or aqueous environment, heavier machine oil, heavier grade lubricating oil; and waste from petrochemical plants, refineries, chemical formulators, pharmaceutical processors, pulp and paper mills, wood processing and treatment plants, metal machining and fabrication plants, distilleries, textiles and food processing.

The particular microbe or microbes may be selected from among those known to have the property to degrade hydrocarbons. Several such microbes are described in the literature and are commercially available for the specific purpose of degrading hydrocarbons such as petroleum products. There are also many types of soil contaminants which can be treated. The microbial content may vary and again, is within the skill of those knowledgeable in the art to use a suitable concentration for a given condition. In a preferred embodiment, a concentrate with a viable bacterial content (CFU) in the billions of organisms per gram may be utilized. In particular embodiments, the concentrate of the compositions disclosed herein may preferably form between 0.5% to 5% by weight of the composition and with a microbial content in excess of 50,000 CFU per gram. The various strains of microorganisms can degrade and detoxify a large range of substituted and unsubstituted aliphatic and aromatic hydrocarbons. In a more particular embodiment, the one or more microbes will be present in a quantity between 1×10² and 1×10¹² CFU/g of the composition, particularly 1×10⁴ and 1×10¹¹ CFU/g of the composition, and more particularly 1×10⁵ and 5×10¹⁰ CFU/g of the composition. In a more particular embodiment the one or more bacterial strains will be present in a quantity between 1×10⁵ and 1×10¹⁰ CFU/g of the composition.

Examples of microbes capable of hydrocarbon degradation (i.e., one or more microbes capable of hydrocarbon degradation) may include one or more bacterial strains selected from the genera consisting of Achromobacter, Acetonema, Actinobacter, Alcaligenes, Alkalibacillus, Ammoniphilus, Amphibacillus, Anaerobacter, Anaerospora, Aneurinibacillus, Anoxybacillus, Arthrobacter, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Enterobacter, Filifactor, Filobacillus, Flavobacterium, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Pseudomonas, Propionispora, Rhodococcus, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof.

In a particular embodiment, the microbes may be selected from those known in the art. Such may include microorganisms of the genus Achromobacter, Actinobacter, Alcaligenes, Arthrobacter, Bacillus, Brevibacillus, Enterobacter, Flavobacterium, Paenibacillus, Pseudomonas, Rhodococcus, and mixtures thereof, and other types of microbes from many different strains. Particularly preferred are those naturally occurring non toxigenic microorganisms of the genus Bacillus, species subtilis, amyloliqueifaciens, licheniformis, and polymyxa.

In another embodiment, the one or more strains capable of hydrocarbon degradation is a strain of Achromobacter spp., e.g., Achromobacter denitrificans; Achromobacter insolitus; Achromobacter piechaudii; Achromobacter ruhlandii, Achromobacter spanius, Achromobacter xylosoxidans, or combinations thereof.

In another embodiment, the one or more strains capable of hydrocarbon degradation is a strain of Alcaligenes spp., e.g., Alcaligenes aquatilis; Alcaligenes eutrophus; Alcaligenes faecalis; Alcaligenes latus, Alcaligenes xylosoxidans, or combinations thereof.

In another embodiment, the one or more strains capable of hydrocarbon degradation is a strain of Arthrobacter spp., e.g., Arthrobacter globiformis; Arthrobacter nicotianae; Arthrobacter chlorophenolicus, or combinations thereof.

In another embodiment, the one or more strains capable of hydrocarbon degradation is a strain of Bacillus spp., e.g., Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firm us, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus, mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, or combinations thereof.

In another embodiment, the one or more strains capable of hydrocarbon degradation is a strain of Brevibacillus spp., e.g., Brevibacillus brevis; Brevibacillus formosus; Brevibacillus laterosporus; or Brevibacillus parabrevis, and combinations thereof.

In another embodiment, the one or more strains capable of hydrocarbon degradation is a strain of Enterobacter spp., e.g., Enterobacter aerogenes; Enterobacter amnigenus; Enterobacter asburiae; Enterobacter cancerogenus; Enterobacter cloacae; Enterobacter cowanii; Enterobacter dissolvens; Enterobacter gergoviae; Enterobacter hormaechei; Enterobacter intermedius; Enterobacter kobei; Enterobacter nimipressuralis; Enterobacter pyrinus; Enterobacter sakazakii, or combinations thereof.

In another embodiment, the one or more strains capable of hydrocarbon degradation is a strain of Flavobacterium spp., e.g., Flavobacterium columnare; Flavobacterium psychrophilum; Flavobacterium branchiophilum, Flavobacterium aquatile; Flavobacterium ferrugineum; Flavobacterium johnsoniae; Flavobacterium limicola; Flavobacterium micromati; Flavobacterium psychrolimnae, or combinations thereof.

In another embodiment, the one or more strains capable of hydrocarbon degradation is a strain of Paenibacillus spp., e.g., Paenibacillus alvei; Paenibacillus amylolyticus; Paenibacillus azotofixans; Paenibacillus cookii; Paenibacillus macerans; Paenibacillus polymyxa; or Paenibacillus validus, and combinations thereof.

In another embodiment, the one or more strains capable of hydrocarbon degradation is a strain of Pseudomonas spp., e.g., Pseudomonas abietaniphila; Pseudomonas agarici; Pseudomonas agarolyticus; Pseudomonas alcaliphila; Pseudomonas alginovora; Pseudomonas andersonii; Pseudomonas antarctica; Pseudomonas asplenii; Pseudomonas azelaica; Pseudomonas batumici; Pseudomonas borealis; Pseudomonas brassicacearum; Pseudomonas chloritidismutans; Pseudomonas cremoricolorata; Pseudomonas diterpeniphila; Pseudomonas filiscindens; Pseudomonas frederiksbergensis; Pseudomonas gingeri; Pseudomonas graminis; Pseudomonas grimontii; Pseudomonas halodenitrificans; Pseudomonas halophila; Pseudomonas hibiscicola; Pseudomonas hydrogenovora; Pseudomonas indica; Pseudomonas japonica; Pseudomonas jessenii; Pseudomonas kilonensis; Pseudomonas koreensis; Pseudomonas lini; Pseudomonas lurida; Pseudomonas lutea; Pseudomonas marginata; Pseudomonas meridiana; Pseudomonas mesoacidophila; Pseudomonas pachastrellae; Pseudomonas palleroniana; Pseudomonas parafulva; Pseudomonas pavonanceae; Pseudomonas proteolyica; Pseudomonas psychrophila; Pseudomonas psychrotolerans; Pseudomonas pudica; Pseudomonas rathonis; Pseudomonas reactans; Pseudomonas rhizosphaerae; Pseudomonas salmononii; Pseudomonas thermaerum; Pseudomonas thermocarboxydovorans; Pseudomonas thermotolerans; Pseudomonas thivervalensis; Pseudomonas umsongensis; Pseudomonas vancouverensis; Pseudomonas wisconsinensis; Pseudomonas xanthomarina; Pseudomonas xiamenensis; Pseudomonas aeruginosa; Pseudomonas alcaligenes; Pseudomonas anguilliseptica; Pseudomonas citronellolis; Pseudomonas f/avescens; Pseudomonas jinjuensis; Pseudomonas mendocina; Pseudomonas nitroreducens; Pseudomonas oleovorans; Pseudomonas pseudoalcaligenes; Pseudomonas resinovorans; Pseudomonas straminae; Pseudomonas aurantiaca; Pseudomonas chlororaphis; Pseudomonas fragi; Pseudomonas lundensis; Pseudomonas taetrolens; Pseudomonas azotoformans; Pseudomonas brenneri; Pseudomonas cedrina; Pseudomonas congelans; Pseudomonas corrugata; Pseudomonas costantinii; Pseudomonas extremorientalis; Pseudomonas fluorescens; Pseudomonas fulgida; Pseudomonas gessardii; Pseudomonas libanensis; Pseudomonas mandelii; Pseudomonas marginalis; Pseudomonas mediterranea; Pseudomonas migulae; Pseudomonas mucidolens; Pseudomonas orientalis; Pseudomonas poae; Pseudomonas rhodesiae; Pseudomonas synxantha; Pseudomonas tolaasii; Pseudomonas trivialis; Pseudomonas veronii; Pseudomonas denitrificans; Pseudomonas pertucinogena; Pseudomonas fulva; Pseudomonas monteilii; Pseudomonas mosselii; Pseudomonas oryzihabitans; Pseudomonas plecoglossicida; Pseudomonas putida; Pseudomonas balearica; Pseudomonas luteola; Pseudomonas stutzeri; Pseudomonas avellanae; Pseudomonas cannabina; Pseudomonas caricapapyae; Pseudomonas cichorii; Pseudomonas coronafaciens; Pseudomonas fuscovaginae; Pseudomonas tremae; Pseudomonas viridiflava, or combinations thereof.

In another embodiment, the one or more strains capable of hydrocarbon degradation is a strain of Rhodococcus spp., e.g., Rhodococcus baikonurensus; Rhodococcus boritolerans; Rhodococcus equius; Rhodococcus corophilus; Rhodococcus corynebacterioides; Rhodococcus erythropolis; Rhodococcus fascians; Rhodococcus globerulus; Rhodococcus gordoniae; Rhodococcus jostii; Rhodococcus jostii RHA 1; Rhodococcus koreensis; Rhodococcus kroppenstedtii; Rhodococcus maanshanensis; Rhodococcus marinonascens; Rhodococcus opacus; Rhodococcus percolatus; Rhodococcus phenolicus; Rhodococcus polyvorum; Rhodococcus pyridinivorans; Rhodococcus rhodochrous; Rhodococcus rhodnii; Rhodococcus ruber; Rhodococcus triatomae; Rhodococcus tukisamuensis; Rhodococcus wratislaviensis; Rhodococcus yunnanensis; or Rhodococcus zopfii, or combinations thereof.

In a more particular embodiment, the one or more strains capable of hydrocarbon degradation comprises one or more strains of Bacillus, one or more strains of Brevibacillus, one or more strains of Paenibacillus, one or more strains of Enterobacter, one or more strains of Rhodococcus, and one or more strains of Pseudomonas.

In still a more particular embodiment, the one or more strains capable of hydrocarbon degradation comprises one or more strains of Bacillus subtilis, one or more strains of Bacillus amyloliquefaciens, one or more strains of Bacillus megaterium, one or more strains of Bacillus licheniformis, one or more strains of Bacillus pumilus, one or more strains of Brevibacillus parabrevis, one or more strains of Enterobacter dissolvens, one or more strains of Paenibacillus validus, one or more strains of Pseudomonas monteilii, one or more strains of Pseudomonas plecoglossicida, one or more strains of Pseudomonas putida, one or more strains of Rhodococcus erythropolis, and one or more strains of Rhodococcus pyridinivorans.

In still another embodiment, the particular microbe or microbes is selected from among those which may have dust suppressing properties. Again, depending on the microbial content, the concentrations may vary, and it is within the skill of those knowledgeable in the art to use a suitable concentration of one or more of the aforementioned microbes for a given condition, such as dust suppression. In a preferred embodiment, a concentrate with a viable bacterial content (CFU) in the billions of organisms per gram may be utilized. In particular embodiments, the concentrate of the compositions disclosed herein may preferably form between 0.5% to 5% by weight of the composition and with a microbial content in excess of 50,000 CFU per gram. In a more particular embodiment, the one or more dust suppressing microbes will be present in a quantity between 1×10² and 1×10¹² CFU/g of the composition, particularly 1×10⁴ and 1×10¹¹ CFU/g of the composition, and more particularly 1×10⁵ and 5×10¹⁰ CFU/g of the composition. In a more particular embodiment the one or more dust suppressing microbes may be present in a quantity between 1×10⁵ and 1×10¹⁰ CFU/g of the composition.

Examples of microbes capable of having dust suppressing properties (i.e., one or more microbes capable of dust suppression) may include one or more bacterial strains selected from the genera consisting of Achromobacter, Acetonema, Actinobacter, Alcaligenes, Alkalibacillus, Ammoniphilus, Amphibacillus, Anaerobacter, Anaerospora, Aneurinibacillus, Anoxybacillus, Arthrobacter, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Enterobacter, Filifactor, Filobacillus, Flavobacterium, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Pseudomonas, Propionispora, Rhodococcus, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, Vulcanobacillus, and combinations thereof.

In another embodiment, the one or more strains capable of dust suppression is a strain of Achromobacter spp., e.g., Achromobacter denitrificans; Achromobacter insolitus; Achromobacter piechaudii; Achromobacter ruhlandii, Achromobacter spanius, Achromobacter xylosoxidans, or combinations thereof.

In another embodiment, the one or more strains capable of dust suppression is a strain of Alcaligenes spp., e.g., Alcaligenes aquatilis; Alcaligenes eutrophus; Alcaligenes faecalis; Alcaligenes latus, Alcaligenes xylosoxidans, or combinations thereof.

In another embodiment, the one or more strains capable of dust suppression is a strain of Arthrobacter spp., e.g., Arthrobacter globiformis; Arthrobacter nicotianae; Arthrobacter chlorophenolicus, or combinations thereof.

In another embodiment, the one or more strains capable of dust suppression is a strain of Bacillus spp., e.g., Bacillus alcalophilus, Bacillus alvei, Bacillus aminovorans, Bacillus amyloliquefaciens, Bacillus aneurinolyticus, Bacillus aquaemaris, Bacillus atrophaeus, Bacillus boroniphilius, Bacillus brevis, Bacillus caldolyticus, Bacillus centrosporus, Bacillus cereus, Bacillus circulans, Bacillus coagulans, Bacillus firmus, Bacillus flavothermus, Bacillus fusiformis, Bacillus globigii, Bacillus infernus, Bacillus larvae, Bacillus laterosporus, Bacillus lentus, Bacillus licheniformis, Bacillus megaterium, Bacillus, mesentericus, Bacillus mucilaginosus, Bacillus mycoides, Bacillus natto, Bacillus pantothenticus, Bacillus polymyxa, Bacillus pseudoanthracis, Bacillus pumilus, Bacillus schlegelii, Bacillus sphaericus, Bacillus sporothermodurans, Bacillus stearothermophillus, Bacillus subtilis, Bacillus thermoglucosidasius, Bacillus thuringiensis, Bacillus vulgatis, Bacillus weihenstephanensis, or combinations thereof.

In another embodiment, the one or more strains capable of dust suppression is a strain of Brevibacillus spp., e.g., Brevibacillus brevis; Brevibacillus formosus; Brevibacillus laterosporus; or Brevibacillus parabrevis, and combinations thereof.

In another embodiment, the one or more strains capable of dust suppression is a strain of Enterobacter spp., e.g., Enterobacter aerogenes; Enterobacter amnigenus; Enterobacter asburiae; Enterobacter cancerogenus; Enterobacter cloacae; Enterobacter cowanii; Enterobacter dissolvens; Enterobacter gergoviae; Enterobacter hormaechei; Enterobacter intermedius; Enterobacter kobei; Enterobacter nimipressuralis; Enterobacter pyrinus; Enterobacter sakazakii, or combinations thereof.

In another embodiment, the one or more strains capable of dust suppression is a strain of Flavobacterium spp., e.g., Flavobacterium columnare; Flavobacterium psychrophilum; Flavobacterium branchiophilum, Flavobacterium aquatile; Flavobacterium ferrugineum; Flavobacterium johnsoniae; Flavobacterium limicola; Flavobacterium micromati; Flavobacterium psychrolimnae, or combinations thereof.

In another embodiment, the one or more strains capable of dust suppression is a strain of Paenibacillus spp., e.g., Paenibacillus alvei; Paenibacillus amylolyticus; Paenibacillus azotofixans; Paenibacillus cookii; Paenibacillus macerans; Paenibacillus polymyxa; or Paenibacillus validus, or combinations thereof.

In another embodiment, the one or more strains capable of dust suppression is a strain of Pseudomonas spp., e.g., Pseudomonas abietaniphila; Pseudomonas agarici; Pseudomonas agarolyticus; Pseudomonas alcaliphila; Pseudomonas alginovora; Pseudomonas andersonii; Pseudomonas antarctica; Pseudomonas asplenii; Pseudomonas azelaica; Pseudomonas batumici; Pseudomonas borealis; Pseudomonas brassicacearum; Pseudomonas chloritidismutans; Pseudomonas cremoricolorata; Pseudomonas diterpeniphila; Pseudomonas filiscindens; Pseudomonas frederiksbergensis; Pseudomonas gingeri; Pseudomonas graminis; Pseudomonas grimontii; Pseudomonas halodenitrificans; Pseudomonas halophila; Pseudomonas hibiscicola; Pseudomonas hydrogenovora; Pseudomonas indica; Pseudomonas japonica; Pseudomonas jessenii; Pseudomonas kilonensis; Pseudomonas koreensis; Pseudomonas lini; Pseudomonas lurida; Pseudomonas lutea; Pseudomonas marginata; Pseudomonas meridiana; Pseudomonas mesoacidophila; Pseudomonas pachastrellae; Pseudomonas palleroniana; Pseudomonas parafulva; Pseudomonas pavonanceae; Pseudomonas proteolyica; Pseudomonas psychrophila; Pseudomonas psychrotolerans; Pseudomonas pudica; Pseudomonas rathonis; Pseudomonas reactans; Pseudomonas rhizosphaerae; Pseudomonas salmononii; Pseudomonas thermaerum; Pseudomonas thermocarboxydovorans; Pseudomonas thermotolerans; Pseudomonas thivervalensis; Pseudomonas umsongensis; Pseudomonas vancouverensis; Pseudomonas wisconsinensis; Pseudomonas xanthomarina; Pseudomonas xiamenensis; Pseudomonas aeruginosa; Pseudomonas alcaligenes; Pseudomonas anguilliseptica; Pseudomonas citronellolis; Pseudomonas flavescens; Pseudomonas jinjuensis; Pseudomonas mendocina; Pseudomonas nitroreducens; Pseudomonas oleovorans; Pseudomonas pseudoalcaligenes; Pseudomonas resinovorans; Pseudomonas straminae; Pseudomonas aurantiaca; Pseudomonas chlororaphis; Pseudomonas fragi; Pseudomonas lundensis; Pseudomonas taetrolens; Pseudomonas azotoformans; Pseudomonas brenneri; Pseudomonas cedrina; Pseudomonas congelans; Pseudomonas corrugata; Pseudomonas costantinii; Pseudomonas extremorientalis; Pseudomonas fluorescens; Pseudomonas fulgida; Pseudomonas gessardii; Pseudomonas libanensis; Pseudomonas mandelii; Pseudomonas marginalis; Pseudomonas mediterranea; Pseudomonas migulae; Pseudomonas mucidolens; Pseudomonas orientalis; Pseudomonas poae; Pseudomonas rhodesiae; Pseudomonas synxantha; Pseudomonas tolaasii; Pseudomonas trivialis; Pseudomonas veronii; Pseudomonas denitrificans; Pseudomonas pertucinogena; Pseudomonas fulva; Pseudomonas monteilii; Pseudomonas mosselii; Pseudomonas oryzihabitans; Pseudomonas plecoglossicida; Pseudomonas putida; Pseudomonas balearica; Pseudomonas luteola; Pseudomonas stutzeri; Pseudomonas avellanae; Pseudomonas cannabina; Pseudomonas caricapapyae; Pseudomonas cichorii; Pseudomonas coronafaciens; Pseudomonas fuscovaginae; Pseudomonas tremae; Pseudomonas viridiflava, or combinations thereof.

In another embodiment, the one or more strains capable of dust suppression is a strain of Rhodococcus spp., e.g., Rhodococcus baikonurensus; Rhodococcus boritolerans; Rhodococcus equius; Rhodococcus corophilus; Rhodococcus corynebacterioides; Rhodococcus erythropolis; Rhodococcus fascians; Rhodococcus globerulus; Rhodococcus gordoniae; Rhodococcus jostii; Rhodococcus jostii RHA1; Rhodococcus koreensis; Rhodococcus kroppenstedtii; Rhodococcus maanshanensis; Rhodococcus marinonascens; Rhodococcus opacus; Rhodococcus percolatus; Rhodococcus phenolicus; Rhodococcus polyvorum; Rhodococcus pyridinivorans; Rhodococcus rhodochrous; Rhodococcus rhodnii; Rhodococcus ruber; Rhodococcus triatomae; Rhodococcus tukisamuensis; Rhodococcus wratislaviensis; Rhodococcus yunnanensis; or Rhodococcus zopfii, or combinations thereof.

In a more particular embodiment, the one or more strains capable of dust suppression comprises one or more strains of Bacillus, one or more strains of Brevibacillus, one or more strains of Paenibacillus, one or more strains of Enterobacter, one or more strains of Rhodococcus, and one or more strains of Pseudomonas.

In still a more particular embodiment, the one or more strains capable of dust suppression comprises one or more strains of Bacillus subtilis, one or more strains of Bacillus amyloliquefaciens, one or more strains of Bacillus megaterium, one or more strains of Bacillus licheniformis, one or more strains of Bacillus pumilus, one or more strains of Brevibacillus parabrevis, one or more strains of Enterobacter dissolvens, one or more strains of Paenibacillus validus, one or more strains of Pseudomonas monteilii, one or more strains of Pseudomonas plecoglossicida, one or more strains of Pseudomonas putida, one or more strains of Rhodococcus erythropolis, and one or more strains of Rhodococcus pyridinivorans.

In still a more particular embodiment, the one or more strains capable of hydrocarbon degradation and dust suppression comprises one or more strains of Bacillus, one or more strains of Brevibacillus, one or more strains of Paenibacillus, one or more strains of Enterobacter, one or more strains of Rhodococcus, and one or more strains of Pseudomonas.

In still yet a more particular embodiment, the one or more strains capable of hydrocarbon degradation and dust suppression are selected from the group consisting of one or more strains of Bacillus subtilis, one or more strains of Bacillus amyloliquefaciens, one or more strains of Bacillus megaterium, one or more strains of Bacillus licheniformis, one or more strains of Bacillus pumilus, one or more strains of Brevibacillus parabrevis, one or more strains of Enterobacter dissolvens, one or more strains of Paenibacillus validus, one or more strains of Pseudomonas monteilii, one or more strains of Pseudomonas plecoglossicida, one or more strains of Pseudomonas putida, one or more strains of Rhodococcus erythropolis, and one or more strains of Rhodococcus pyridinivorans, and combinations thereof.

In another particular embodiment, the one or more microbial strains capable of dust suppression, according to invention, are capable of forming biofilm (e.g., biofilm forming bacterial strains, preferably biofilm forming Bacillus species). Biofilms and the capability of forming biofilms are described in O'Toole G. A. (2011), “Microtiter Dish Biofilm Formation Assay”, Journal of Visualized Experiments, 47; http://www.jove.com/details.php?id=2437, doi: 10.3791/2437.

In a most particular embodiment, the one or more microbial strains capable of dust suppression, according to invention, are spores (as opposed to vegetative cells). Preferably, the one or more microbial strains are bacterial spores; more preferably the one or more microbial strains are Bacillus spores; even more preferably the one or more microbial strains are spores of Bacillus subtilis, Bacillus licheniformis, Bacillus amyloliquefaciens, or Bacillus megaterium; even more preferably the one or more microbial strains are spores selected from the group consisting of:
Bacillus subtilis;
Bacillus licheniformis;
Bacillus amyloliquefaciens;
Bacillus megaterium;
Bacillus subtilis, and Bacillus licheniformis;
Bacillus subtilis, and Bacillus amyloliquefaciens;
Bacillus subtilis, and Bacillus megaterium;
Bacillus licheniformis, and Bacillus amyloliquefaciens;
Bacillus licheniformis, and Bacillus megaterium;
Bacillus amyloliquefaciens, and Bacillus megaterium;
Bacillus subtilis, Bacillus licheniformis, and Bacillus amyloliquefaciens;
Bacillus subtilis, Bacillus licheniformis, and Bacillus megaterium;
Bacillus licheniformis, Bacillus amyloliquefaciens, and Bacillus megaterium; and Bacillus subtilis, Bacillus licheniformis, Bacillus amyloliquefaciens, and Bacillus megaterium.

In a particularly preferred embodiment, the above-mentioned Bacillus strains are selected from the group consisting of Bacillus subtilis ATCC 6051A, Bacillus subtilis NRRL B-50622, Bacillus subtilis ATCC 55406, Bacillus subtilis NRRL B-50136, Bacillus licheniformis ATCC 12713, Bacillus licheniformis NRRL B-50623, Bacillus amyloliquifaciens SB3106, Bacillus amyloliquifaciens NRRL B-50147, and Bacillus megaterium ATCC 14581.

The fermentation of the one or more of the microbial strains disclosed herein (e.g., microbial strains capable of dust suppression, microbes capable of hydrocarbon degradation, or microbes capable of dust suppression and hydrocarbon degradation) may be conducted using conventional fermentation processes, such as, aerobic liquid-culture techniques, shake flask cultivation, and small-scale or large-scale fermentation (e.g., continuous, batch, fed-batch, solid state fermentation, etc.) in laboratory or industrial fermentors, and such processes are well known in the art. Notwithstanding the production process used to produce the one or more bacterial strains, the one or more bacterial strains may be used directly from the culture medium or subject to purification and/or further processing steps (e.g., a drying process).

Following fermentation, the one or more bacterial strains may be recovered using conventional techniques (e.g., by filtration, centrifugation, etc.). The one or more bacterial strains may alternatively be dried (e.g., air-drying, freeze drying, or spray drying to a low moisture level, and storing at a suitable temperature, e.g., room temperature).

### Beneficial Ingredients

The compositions disclosed herein may comprise one or more beneficial ingredients. The composition can utilize other materials apart from glycerine, the material having a tackiness which is sufficient to bind dirt particles together. Such materials will include guar, synthetic oils, resins, lignin and lignosulfonates, molasses, carbohydrate based products, glycerine, vegetable based oils, vegetable based oil emulsions, sulfonated oils, non-cross linking carbon based polymers, etc. Non-limiting examples of beneficial ingredients include one or more, polymers, wetting agents, surfactants, or combinations thereof.

### Polymers

In one embodiment, the compositions described herein may further comprise one or more polymers. Polymers for use in the dust suppression are well known. Non-limiting examples of commercial products including polymers used for dust suppression include Dusgon® (DuPont, Austrailia); Soiltac®, Powdered Soiltac®, GorillaSnot®, Durasoil® (Soilworks, Ariz., USA). In one embodiment, the one or more polymers is a natural polymer (e.g., agar, starch, alginate, pectin, cellulose, resins, etc.), a synthetic polymer, a biodegradable polymer (e.g., polyvinyl acetate polycaprolactone, polylactide, poly (vinyl alcohol), etc.), or a combination thereof.

For a non-limiting list of polymers useful for the compositions described herein, see Pouci, et al., Am. J. Agri. & Biol. Sci., 3(1):299-314 (2008). In one embodiment, the compositions described herein comprise non-cross linking carbon based polymers, cellulose, cellulose derivatives, lignins, lignosulfonates (i.e., lignin sulfonates, sulphite lignins, etc.) methylcellulose, methylcellulose derivatives, starch, agar, alginate, pectin, polyvinylpyrrolidone, and combinations thereof.

In still a more particular embodiment, a small amount (less than 10% w/w, preferably less than 5%, more preferably less than 2%, more preferably less than 1%, more preferably less than 0.5%, most preferably less than 0.25%) of a natural polymer is added to the composition. Natural polymers are well known in the art and can be selected from many different such polymers. Polymers are compounds or a mixture of compounds consisting of repeating structural units created through a process called polymerization. Such natural polymers include proteins and nucleic acids, cellulose, starch, etc.

In a particular embodiment, a lignosulfonate may be added to the composition. In a greater detail, the lignins are natural complex polymers which are generally produced as a co-product of the paper industry, the lignins being separated from the trees by a chemical pulping process. Lignosulfonates are also known as lignin sulfonates and sulphite lignins are products of sulphite pulping. Other delignifying technologies may include the use of an organic solvent or high pressure steam treatment to remove lignins from plants.

As aforementioned, lignin is a very complex natural polymer, the exact chemical structure not being known. Physical and chemical properties can differ depending on the extraction technology. Lignosulfonates have typically been used for their dispersing, binding, complexing and emulsifying properties. Lignins have been used for many years and extensive studies have been done to test lignin impact on the environment. To date, lignins have been shown to be safe and not harmful to plants, animals and aquatic life when properly manufactured and applied. Furthermore, lignosulfonates have been found to be essentially non-toxic and non-irritating, non-mutagenic nor toxic and may be widely used in animal and human feed contact products.

Surprisingly, it has been found that the use of the lignosulfonate with the microbes is a very efficient and cost effective way of cleaning hydrocarbon containing substrates and/or suppressing dust. Without being limited to any particular theory, it is thought that the lignosulfonates provide a readily available food source for the microbes and the lignosulfonate also helps in the cleaning. As such, the microbes are in a healthy and active state when they are placed in contact with the hydrocarbons and hence are able to reactivate themselves very quickly and thus are highly effective.

As described above, the microbial content may vary and again, is within the skill of those knowledgeable in the art to use a suitable concentration for a given condition. In a preferred embodiment, a concentrate with a viable bacterial content (CFU) in the billions of organisms per gram may be utilized. After mixing with the lignosulfonate, the concentrate may preferably form between 0.5% to 5% by weight of the composition and with a microbial content in excess of 50,000 CFU per gram. The various strains of microorganisms can degrade and detoxify a large range of substituted and unsubstituted aliphatic and aromatic hydrocarbons.

In a particular embodiment, the water and liquid glycerine make up a substantial portion of the composition with the natural polymer, lignosulfonate, and one or more of the microbes described herein being added in substantially smaller quantities.

In another embodiment, the compositions described herein may further comprise one or more resins (e.g., pine resin, tree resin, amber, etc.). In still another embodiment, the compositions comprises, water, liquid glycerine, lignosulfonate, one or more of the microbes described herein and one or more resins.

### α-Hydroxy Acids (AHAs)

α-Hydroxy acids, or alpha hydroxy acids (AHAs), are a class of chemical compounds that consist of a carboxylic acid substituted with a hydroxyl group on the adjacent carbon. They may be either naturally occurring or synthetic. Non-limiting examples of AHAs include glucolic acid, lactic acid, citric acid, and mandelic acid.

In an embodiment, the compositions described herein may further comprise one or more AHAs. In a particular embodiment, the compositions described herein comprise an AHA selected from the group consisting of glycolic acid, lactic acid, citric acid, mandelic acid, and combinations thereof. In a particular embodiment, the compositions described herein further comprise glycolic acid. In another embodiment the compositions described herein comprise lactic acid. In still yet another embodiment, the compositions described herein comprise glycolic acid and lactic acid.

### Enzymes

One or more enzymes may be present in a composition of the invention. The one or more enzymes may be useful for degrading one or more contaminants (e.g., hydrocarbons). Especially contemplated enzymes include proteases, alpha-amylases, cellulases, lipases, peroxidases/oxidases, pectate lyases, and mannanases, or mixtures thereof.

Proteases: Suitable proteases include those of animal, vegetable or microbial origin. Microbial origin is preferred. Chemically modified or protein engineered mutants are included.

The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274. Preferred commercially available protease enzymes include ALCALASE™, SAVINASE™, PRIMASE™, DURALASE™, DYRAZYM™, ESPERASE™, EVERLASE™, POLARZYME™ and KANNASE™, LIQUANASE™ (Novozymes NS), MAXATASE™, MAXACAL™ MAXAPEM™, PROPERASE™, PURAFECT™, PURAFECT OxP™, FN2™, and FN3™ (Genencor International Inc.).

Lipases: Suitable lipases include those of bacterial or fungal origin. Chemically modified or protein engineered mutants are included. Examples of useful lipases include lipases from Humicola (synonym Thermomyces), e.g., from H. lanuginosa (T. lanuginosus) as described in EP 258 068 and EP 305 216 or from H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta 1131:253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Preferred commercially available lipase enzymes include LIPOLASE™ and LIPOLASE ULTRA™, LIPOZYME™, and LIPEX™ (Novozymes NS).

Cutinase: The method of the invention may be carried out in the presence of cutinase classified in EC 3.1.1.74.

The cutinase used according to the invention may be of any origin. Preferably cutinases are of microbial origin, in particular of bacterial, of fungal or of yeast origin.

Cutinases are enzymes which are able to degrade cutin. In a preferred embodiment, the cutinase is derived from a strain of Aspergillus, in particular Aspergillus oryzae, a strain of Alternaria, in particular Alternaria brassicicola, a strain of Fusarium, in particular Fusarium solani, Fusarium solani pisi, Fusarium roseum culmorum, or Fusarium roseum sambucium, a strain of Helminthosporum, in particular Helminthosporum sativum, a strain of Humicola, in particular Humicola insolens, a strain of Pseudomonas, in particular Pseudomonas mendocina, or Pseudomonas putida, a strain of Rhizoctonia, in particular Rhizoctonia solani, a strain of Streptomyces, in particular Streptomyces scabies, or a strain of Ulocladium, in particular Ulocladium consortiale. In a most preferred embodiment the cutinase is derived from a strain of Humicola insolens, in particular the strain Humicola insolens DSM 1800. Humicola insolens cutinase is described in WO 96/13580 which is hereby incorporated by reference. The cutinase may be a variant, such as one of the variants disclosed in WO 00/34450 and WO 01/92502, which are hereby incorporated by reference. Preferred cutinase variants include variants listed in Example 2 of WO 01/92502, which is hereby specifically incorporated by reference.

Preferred commercial cutinases include NOVOZYM™ 51032 (available from Novozymes NS, Denmark).

The method of the invention may be carried out in the presence of phospholipase classified as EC 3.1.1.4 and/or EC 3.1.1.32. As used herein, the term phospholipase is an enzyme which has activity towards phospholipids. Phospholipids, such as lecithin or phosphatidylcholine, consist of glycerol esterified with two fatty acids in an outer (sn-1) and the middle (sn-2) positions and esterified with phosphoric acid in the third position; the phosphoric acid, in turn, may be esterified to an amino-alcohol. Phospholipases are enzymes which participate in the hydrolysis of phospholipids. Several types of phospholipase activity can be distinguished, including phospholipases Aₗ and A₂ which hydrolyze one fatty acyl group (in the sn-1 and sn-2 position, respectively) to form lysophospholipid; and lysophospholipase (or phospholipase B) which can hydrolyze the remaining fatty acyl group in lysophospholipid. Phospholipase C and phospholipase D (phosphodiesterases) release diacyl glycerol or phosphatidic acid respectively.

The term phospholipase includes enzymes with phospholipase activity, e.g., phospholipase A (A₁ or A₂), phospholipase B activity, phospholipase C activity or phospholipase D activity. The term “phospholipase A” used herein in connection with an enzyme of the invention is intended to cover an enzyme with Phospholipase A₁ and/or Phospholipase A₂ activity. The phospholipase activity may be provided by enzymes having other activities as well, such as, e.g., a lipase with phospholipase activity. The phospholipase activity may, e.g., be from a lipase with phospholipase side activity. In other embodiments of the invention the phospholipase enzyme activity is provided by an enzyme having essentially only phospholipase activity and wherein the phospholipase enzyme activity is not a side activity.

The phospholipase may be of any origin, e.g., of animal origin (such as, e.g., mammalian), e.g., from pancreas (e.g., bovine or porcine pancreas), or snake venom or bee venom. Preferably the phospholipase may be of microbial origin, e.g., from filamentous fungi, yeast or bacteria, such as the genus or species Aspergillus, e.g., A. niger, Dictyostelium, e.g., D. discoideum; Mucor, e.g., M. javanicus, M. mucedo, M. subtilissimus; Neurospora, e.g., N. crassa; Rhizomucor, e.g., R. pusillus; Rhizopus, e.g., R. arrhizus, R. japonicus, R. stolonifer, Sclerotinia, e.g., S. libertiana; Trichophyton, e.g., T. rubrum; Whetzelinia, e.g., W. sclerotiorum; Bacillus, e.g., B. megaterium, B. subtilis; Citrobacter, e.g., C. freundii; Enterobacter, e.g., E. aerogenes, E. cloacae; Edwardsiella, E. tarda; Erwinia, e.g., E. herbicola; Escherichia, e.g., E. coli; Klebsiella, e.g., K. pneumoniae; Proteus, e.g., P. vulgaris; Providencia, e.g., P. stuartii; Salmonella, e.g., S. typhimurium; Serratia, e.g., S. liquefasciens, S. marcescens; Shigella, e.g., S. flexneri; Streptomyces, e.g., S. violeceoruber, Yersinia, e.g., Y. enterocolitica. Thus, the phospholipase may be fungal, e.g., from the class Pyrenomycetes, such as the genus Fusarium, such as a strain of F. culmorum, F. heterosporum, F. solani, or a strain of F. oxysporum. The phospholipase may also be from a filamentous fungus strain within the genus Aspergillus, such as a strain of Aspergillus awamori, Aspergillus foetidus, Aspergillus japonicus, Aspergillus niger or Aspergillus oryzae.

Preferred phospholipases are derived from a strain of Humicola, especially Humicola lanuginosa. The phospholipase may be a variant, such as one of the variants disclosed in WO 00/32758, which are hereby incorporated by reference. Preferred phospholipase variants include variants listed in Example 5 of WO 00/32758, which is hereby specifically incorporated by reference. In another preferred embodiment the phospholipase is one described in WO 04/111216, especially the variants listed in the table in Example 1.

In another preferred embodiment the phospholipase is derived from a strain of Fusarium, especially Fusarium oxysporum. The phospholipase may be the one concerned in WO 98/026057 displayed in SEQ ID NO:2 derived from Fusarium oxysporum DSM 2672, or variants thereof.

In a preferred embodiment of the invention the phospholipase is a phospholipase A₁ (EC. 3.1.1.32). In another preferred embodiment of the invention the phospholipase is a phospholipase A₂ (EC.3.1.1.4.).

Examples of commercial phospholipases include LECITASE™ and LECITASE™ ULTRA, YIELSMAX, or LIPOPAN F (available from Novozymes NS, Denmark).

Amylases: Suitable amylases (alpha and/or beta) include those of bacterial or fungal origin. Chemically modified or protein engineered mutants are included. Amylases include, for example, alpha-amylases obtained from Bacillus, e.g., a special strain of B. licheniformis, described in more detail in GB 1,296,839, or the Bacillus sp. strains disclosed in WO 95/026397 or WO 00/060060.

Examples of useful amylases are the variants described in WO 94/02597, WO 94/18314, WO 96/23873, WO 97/43424, WO 01/066712, WO 02/010355, WO 02/031124 and WO 2006/002643 (which references all incorporated by reference).

Commercially available amylases are DURAMYL™, TERMAMYL™, TERMAMYL ULTRA™, NATALASE™, STAINZYME™, STAINZYME ULTRA™, FUNGAMYL™ and BAN™ (Novozymes NS), RAPIDASE™ and PURASTAR™ (from Genencor International Inc.).

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Thielavia terrestris, Myceliophthora thermophila, and Fusarium oxysporum disclosed in U.S. Pat. No. 4,435,307, U.S. Pat. No. 5,648,263, U.S. Pat. No. 5,691,178, U.S. Pat. No. 5,776,757, WO 89/09259, WO 96/029397, and WO 98/012307.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO 1999/001544.

Commercially available cellulases include CELLUZYME™, CELLUCLAST™, CAREZYME™, ENDOLASE™, RENOZYME™ (Novozymes NS), CLAZINASE™ and PURADAX HA™, ACCELERASE™ 1000 (Genencor International Inc.), and KAC-500(B)™ (Kao Corporation).

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzym™ and Novozym™ 51004 (Novozymes A/S).

Pectate lyases (also called polygalacturonate lyases): Examples of pectate lyases include pectate lyases that have been cloned from different bacterial genera such as Erwinia, Pseudomonas, Klebsiella and Xanthomonas, as well as from Bacillus subtilis (Nasser et al., 1993, FEBS Letts. 335:319-326) and Bacillus sp. YA-14 (Kim et al., 1994, Biosci. Biotech. Biochem. 58: 947-949). Purification of pectate lyases with maximum activity in the pH range of 8-10 produced by Bacillus pumilus (Dave and Vaughn, 1971, J. Bacteriol. 108: 166-174), B. polymyxa (Nagel and Vaughn, 1961, Arch. Biochem. Biophys. 93:344-352), B. stearothermophilus (Karbassi and Vaughn, 1980, Can. J. Microbiol. 26: 377-384), Bacillus sp. (Hasegawa and Nagel, 1966, J. Food Sci. 31: 838-845) and Bacillus sp. RK9 (Kelly and Fogarty, 1978, Can. J Microbiol. 24:1164-1172) have also been described. Any of the above, as well as divalent cation-independent and/or thermostable pectate lyases, may be used in practicing the invention. In preferred embodiments, the pectate lyase comprises the amino acid sequence of a pectate lyase disclosed in Heffron et al., 1995, Mol. Plant-Microbe Interact. 8: 331-334 and Henrissat et al., 1995, Plant Physiol. 107: 963-976. Specifically contemplated pectate lyases are disclosed in WO 99/27083 and WO 99/27084. Other specifically contemplated pectate lyases derived from Bacillus licheniformis is disclosed as SEQ ID NO: 2 in U.S. Pat. No. 6,284,524 (which document is hereby incorporated by reference). Specifically contemplated pectate lyase variants are disclosed in WO 02/006442, especially the variants disclosed in the Examples in WO 02/006442 (which document is hereby incorporated by reference).

Examples of commercially available alkaline pectate lyases include BIOPREP™ and SCOURZYME™ L from Novozymes NS, Denmark.

Mannanase: Examples of mannanases (EC 3.2.1.78) include mannanases of bacterial and fungal origin. In a specific embodiment the mannanase is derived from a strain of the filamentous fungus genus Aspergillus, preferably Aspergillus niger or Aspergillus aculeatus (WO 94/25576). WO 93/24622 discloses a mannanase isolated from Trichoderma reesei.

Mannanases have also been isolated from several bacteria, including Bacillus organisms. For example, Talbot et al., 1990, Appl. Environ. Microbiol. 56(11): 3505-3510 describes a beta-mannanase derived from Bacillus stearothermophilus. Mendoza et al., 1994, World J. Microbiol. Biotech. 10(5): 551-555 describes a beta-mannanase derived from Bacillus subtilis. JP-A-03047076 discloses a beta-mannanase derived from Bacillus sp. JP-A-63056289 describes the production of an alkaline, thermostable beta-mannanase. JP-A-63036775 relates to the Bacillus microorganism FERM P-8856 which produces beta-mannanase and beta-mannosidase. JP-A-08051975 discloses alkaline beta-mannanases from alkalophilic Bacillus sp. AM-001. A purified mannanase from Bacillus amyloliquefaciens is disclosed in WO 97/11164. WO 91/18974 describes a hemicellulase such as a glucanase, xylanase or mannanase active. Contemplated are the alkaline family 5 and 26 mannanases derived from Bacillus agaradhaerens, Bacillus licheniformis, Bacillus halodurans, Bacillus clausii, Bacillus sp., and Humicola insolens disclosed in WO 99/64619. Especially contemplated are the Bacillus sp. mannanases concerned in the Examples in WO 99/64619 which document is hereby incorporated by reference.

Examples of commercially available mannanases include MANNAWAY™ available from Novozymes NS Denmark.

### Stabilizers

If one or more enzymes is/are present in the composition it/they may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in e.g., WO 92/19709 and WO 92/19708.

### Wetting Agent(s)

In one embodiment, the compositions described herein may further comprise one or more wetting agents. Wetting agents are commonly used on soils, particularly hydrophobic soils, to obtain controlled infiltration and/or penetration properties into a soil. The wetting agent may be an adjuvant, oil, surfactant, buffer, acidifier, or combination thereof. In an embodiment, the wetting agent is a surfactant. In an embodiment, the wetting agent is one or more nonionic surfactants, one or more anionic surfactants, or a combination thereof. In yet another embodiment, the wetting agent is one or more nonionic surfactants.

Surfactants suitable for the compositions described herein are provided in the “Surfactants” section.

### Surfactant(s)

Surfactants suitable for the compositions described herein may be non-ionic surfactants (e.g., semi-polar and/or anionic and/or cationic and/or zwitterionic). The surfactants can wet and emulsify soil(s) and/or dirt(s). It is envisioned that the surfactants used in the composition described herein have low toxicity for any microorganisms contained within the formulation. It is further envisioned that the surfactants used in the described composition have a low phytotoxicity (i.e., the degree of toxicity a substance or combination of substances has on a plant). A single surfactant or a blend of several surfactants can be used.

### Anionic Surfactants

Anionic surfactants or mixtures of anionic and nonionic surfactants may also be used in the compositions. Anionic surfactants are surfactants having a hydrophilic moiety in an anionic or negatively charged state in aqueous solution. The compositions described herein may comprise one or more anionic surfactants. The anionic surfactant(s) may be either water soluble anionic surfactants, water insoluble anionic surfactants, or a combination of water soluble anionic surfactants and water insoluble anionic surfactants. Non-limiting examples of anionic surfactants include sulfonic acids, sulfuric acid esters, carboxylic acids, and salts thereof. Non-limiting examples of water soluble anionic surfactants include alkyl sulfates, alkyl ether sulfates, alkyl amido ether sulfates, alkyl aryl polyether sulfates, alkyl aryl sulfates, alkyl aryl sulfonates, monoglyceride sulfates, alkyl sulfonates, alkyl amide sulfonates, alkyl aryl sulfonates, benzene sulfonates, toluene sulfonates, xylene sulfonates, cumene sulfonates, alkyl benzene sulfonates, alkyl diphenyloxide sulfonate, alpha-olefin sulfonates, alkyl naphthalene sulfonates, paraffin sulfonates, lignin sulfonates, alkyl sulfosuccinates, ethoxylated sulfosuccinates, alkyl ether sulfosuccinates, alkylamide sulfosuccinates, alkyl sulfosuccinamate, alkyl sulfoacetates, alkyl phosphates, phosphate ester, alkyl ether phosphates, acyl sarconsinates, acyl isethionates, N-acyl taurates, N-acyl-N-alkyltaurates, alkyl carboxylates, or a combination thereof.

### Nonionic Surfactants

Nonionic surfactants are surfactants having no electrical charge when dissolved or dispersed in an aqueous medium. In at least one embodiment of the composition described herein, one or more nonionic surfactants are used as they provide the desired wetting and emulsification actions and do not significantly inhibit spore stability and activity. The nonionic surfactant(s) may be either water soluble nonionic surfactants, water insoluble nonionic surfactants, or a combination of water soluble nonionic surfactants and water insoluble nonionic surfactants.

### Water Insoluble Nonionic Surfactants

Non-limiting examples of water insoluble nonionic surfactants include alkyl and aryl:

glycerol ethers, glycol ethers, ethanolamides, sulfoanylamides, alcohols, amides, alcohol ethoxylates, glycerol esters, glycol esters, ethoxylates of glycerol ester and glycol esters, sugar-based alkyl polyglycosides, polyoxyethylenated fatty acids, alkanolamine condensates, alkanolamides, tertiary acetylenic glycols, polyoxyethylenated mercaptans, carboxylic acid esters, polyoxyethylenated polyoxyproylene glycols, sorbitan fatty esters, or combinations thereof. Also included are EO/PO block copolymers (EO is ethylene oxide, PO is propylene oxide), EO polymers and copolymers, polyamines, and polyvinylpynolidones.

### Water Soluble Nonionic Surfactants

Non-limiting examples of water soluble nonionic surfactants include sorbitan fatty acid alcohol ethoxylates and sorbitan fatty acid ester ethoxylates.

### Combination of Nonionic Surfactants

In one embodiment, the compositions described herein comprise at least one or more nonionic surfactants. In one embodiment, the compositions comprise at least one water insoluble nonionic surfactant and at least one water soluble nonionic surfactant. In still another embodiment, the compositions comprise a combination of nonionic surfactants having hydrocarbon chains of substantially the same length.

### Other Surfactants

In another embodiment, the compositions described herein may also comprise organosilicone surfactants, silicone-based antifoams used as surfactants in silicone-based and mineral-oil based antifoams. In yet another embodiment, the compositions described herein may also comprise alkali metal salts of fatty acids (e.g., water soluble alkali metal salts of fatty acids and/or water insoluble alkali metal salts of fatty acids).

### Anti-Freezing Agent(s)

In one embodiment, the compositions described herein may further comprise one or more anti-freezing agents. Non-limiting examples of anti-freezing agents include ethylene glycol, propylene glycol, urea, glycerin, and combinations thereof.

### Methods

In another aspect, methods of using microorganisms for dust suppression, hydrocarbon degradation, or both are disclosed. In a particular embodiment, the method includes suppressing dust comprising applying to a substrate one or more microorganisms described herein (e.g., microorganisms capable of suppressing dust, microbes capable of hydrocarbon degradation, etc.). In a particular embodiment, the applying step includes applying to a road, surface (e.g., a high traffic surface such as a path or trail, regardless of the ability of the surface to support a motorized vehicular traffic) or substrate comprising dust or dirt (e.g., a mining road, a construction site, trail path, racetrack, animal racing surface, such as a horse track, stockpiles, dumping areas, landfills, etc.; namely, any area in need of dust suppression) one or more of the compositions described herein.

In yet another embodiment, the method includes degrading hydrocarbons comprising applying to a substrate one or more microorganisms described herein. In a particular embodiment, the applying step includes applying to a road, surface or substrate comprising one or more hydrocarbons (e.g., a mining road, a construction site, trail path, racetrack, animal racing surface, such as a horse track, etc.) one or more of the compositions described herein.

In yet another embodiment, the method includes suppressing dust and degrading hydrocarbons comprising applying to a substrate one or more microorganisms described herein. In a particular embodiment, the applying step includes applying to a road, surface or substrate comprising dust or dirt and one or more hydrocarbons (e.g., a mining road, a construction site, trail path, racetrack, animal racing surface, such as a horse track, etc.) one or more of the compositions described herein.

The application of the composition to the road surface is important, when treatment of a road is contemplated. The road surface may be graded and then may have water applied thereto. The surface is preferably compacted following the application of the water. Subsequently, the composition will be applied to the road surface. Preferably, the road should not have any traffic for approximately 24 hours after application of the composition. The applying step can be performed by any method known in the art. Non-limiting examples of applying to the road, surface, or substrate comprising dust include spraying (e.g., a spray bar and pump, etc.), drenching, or dripping onto a road, surface, or substrate comprising dust. In a more particular embodiment, the applying step is repeated (e.g., more than once, as in the contacting step is repeated twice, three times, four times, five times, six times, seven times, eight times, nine times, ten times, etc.).

In an embodiment, the composition is applied to the road using a spray bar and a pump. The application is generally at a rate of between 5,000 to 10,000 gallons per km of road surface (although this can vary depending on road bed composition). The application needs to be made to thoroughly cover the road. Generally, three to four applications per year would suffice to fully suppress the dust. The applying step can occur at any time dust needs to be suppressed, hydrocarbons need to be degraded, or both. In one embodiment, the applying step occurs after particles of dirt/dust have become suspended in the air. In yet another embodiment, the applying step occurs before particles of dirt/dust are suspended in the air. In still another embodiment, the applying step occurs after hydrocarbons have contaminated a road, surface, or substrate. In still yet another embodiment, the applying step occurs before hydrocarbons have contaminated a road, surface, or substrate. In a preferred embodiment, the applying step occurs after particles of dirt/dust have become suspended in the air and after hydrocarbons have contaminated a road surface or substrate. In another preferred embodiment, the applying step occurs before particles of dirt/dust have become suspended in the air and after hydrocarbons have contaminated a road surface or substrate. In still another preferred embodiment, the applying step occurs after particles of dirt/dust have become suspended in the air and before hydrocarbons have contaminated a road surface or substrate. In still yet another preferred embodiment, the applying step occurs before particles of dirt/dust have become suspended in the air and before hydrocarbons have contaminated a road surface or substrate.

In an embodiment, the soil surface, road surface or substrate retains reduced dust formation after a month, preferably two months, more preferably three months, from applying the composition of the invention to the soil surface, road surface or substrate.

### EXAMPLES

The following examples are provided for illustrative purposes and are not intended to limit the scope of the invention as claimed herein. Any variations in the exemplified examples which occur to the skilled artisan are intended to fall within the scope of the present invention.

The microbial strains used in the examples were equal amounts of Bacillus subtilis NRRL B-50622, Bacillus subtilis NRRL B-50136, Bacillus licheniformis NRRL B-50623, Bacillus amyloliquifaciens SB3106, and Bacillus megaterium ATCC 14581. All strains are available from Novozymes.

Unless otherwise indicated, chemicals, buffers and substrates were commercial products of at least reagent grade.

### Example 1

### Field Trial I

The composition was applied to the road using a spray bar and a pump. In the conditions tested, the application was made at a rate of between 5,000 to 10,000 gallons per km of road surface (although this can vary depending on road bed composition). The application was made to thoroughly cover the road. Generally, three to four applications per year would suffice to fully suppress the dust.

A dust suppressant formula was mixed according to the following formulation:

| | Amount by weight |
|---|---|
| Technical grade glycerine | 46% |
| Lignosulfonate polymer | 2% |
| Dust suppressing microbes | 2% |
| Water | up to 100% |

The dust suppressing microbes were a mixture of spores of Bacillus subtilis, Bacillus licheniformis, Bacillus amyloliquefaciens, and Bacillus megaterium. The final total spore concentration was approx. 3×10⁷ CFU/mL.

The objective of the field test performed from Mar. 26, 2012 to Apr. 22, 2012 was to evaluate the ability and effectiveness of the dust suppressant to control dust emission at a mine site (at an elevation of 3,800 to 5,200 meters). The dust emission baseline was established and the dust suppressant was applied on a 200 m×33 m surface of a mine haul road. The dust emission following the application of the dust suppressant was monitored about a week (8 days) after the application of the dust suppressant, and almost a month (25 days) after the application.

### Dust Monitor

Real-time dust monitoring was performed using a DUSTTRAK™ DRX Aerosol Monitor 8533 which can simultaneously measure size-segregated mass fraction concentrations corresponding to PM₁, PM₂₅, Respirable, PM₁₀ and Total PM size fractions. The aerosol concentration range of the monitor is 0.001 to 150 mg/m³ (or ppm).

### Dust Emission Measurements Performed

Dust emission was measured using two different types of measurement:
1. Mobile monitoring—having the monitor installed at the back of a pickup truck as described in Table 1.
2. Static monitoring—having a mine truck passing at about 1.5 to 2.5 meters of the monitor located on the side of the road, and described in Table 1.

The conditions used for each type of measurement are summarized in Table 1 and the characteristics of the roads are summarized in Table 2.

TABLE 1
Types of measurement.

| Type of | Type of | Driving | |
|---|---|---|---|
| measurement | truck | speed | Position of dust monitor |
| Mobile | Pickup truck | 40 km/h | At the back of the pick-up |
| monitoring | | | truck and connected to the |
| | | | sampling tube |
| Static | Mining truck | 30 km/h | At the back of the pick up |
| monitoring | KMS930E | | truck parked perpendicular |
| | fully | | to road (100 m from stop |
| | loaded | | sign) and located at 1.5- |
| | | | 2.5 m from the mining truck |

TABLE 2
Description of roads monitored.

| | |
|---|---|
| Dry mine | Altitude 5100 m |
| road | Mine road between the median and open pit |
| | Dust emission monitored using static dust monitoring |
| Wet mine | Altitude 5100 m |
| road | Mine road beside the dry road (on the other side of |
| | median) |
| | Sprayed with water hours prior to dust measurement |
| | Dust emission monitored using static & mobile dust |
| | monitoring |
| Treated | Altitude 5100 m |
| mine road | Same as wet mine road |
| | Treated with the dust suppressant |
| | Dust emission monitored using static & mobile dust |
| | monitoring |
| Service | Zigzag road |
| roads | Dust emission monitored using mobile dust monitoring |

### Results

The results of the measurements are shown in Table 3 and in the accompanying figures.

TABLE 3
Dust measurements in ppm with mean particle sizes.

| | Mobile dust monitoring | | Static dust monitoring | |
|---|---|---|---|---|
| | Average | Maximum | Average | Maximum |
| | dust con- | dust con- | dust con- | dust con- |
| | centration | centration | centration | centration |
| Road | (ppm) | (ppm) | (ppm) | (ppm) |
| Dry mine | PM_{2.5}: 51 | PM_{2.5}: 150 | PM_{2.5}: 9 | PM_{2.5}: 36 |
| road | PM₁₀: 68 | PM₁₀: 150 | PM₁₀: 17 | PM₁₀: 59 |
| | Total: 72 | Total: 150 | Total: 22 | Total: 81 |
| Wet mine | PM_{2.5}: 15 | PM_{2.5}: 35 | PM_{2.5}: 5 | PM_{2.5}: 13 |
| road | PM₁₀: 18 | PM₁₀: 40 | PM₁₀: 8 | PM₁₀: 19 |
| | Total: 21 | Total: 43 | Total: 10 | Total: 26 |
| Treated | PM_{2.5}: 0.2 | PM_{2.5}: 1.1 | PM_{2.5}: 0.1 | PM_{2.5}: 0.5 |
| mine road | PM₁₀: 0.3 | PM₁₀: 1.8 | PM₁₀: 0.2 | PM₁₀: 0.7 |
| (April 5) | Total: 0.3 | Total: 2.2 | Total: 0.2 | Total: 0.8 |
| Treated | PM_{2.5}: 1.9 | PM_{2.5}: 14 | — | — |
| mine road | PM₁₀: 2.4 | PM₁₀: 15 | | |
| (April 22) | Total: 3.0 | Total: 17.3 | | |

### Data Analysis

For the static dust monitoring, the average concentration was calculated using the values stored by the monitor every second during the passage of the mine truck close to the monitor located on the side of the road. For the mobile dust monitoring, the average dust concentration was calculated using the value stored by the monitor every second during the time the pickup truck was driven at 40 km/h.

### Monitoring of Dust Emission

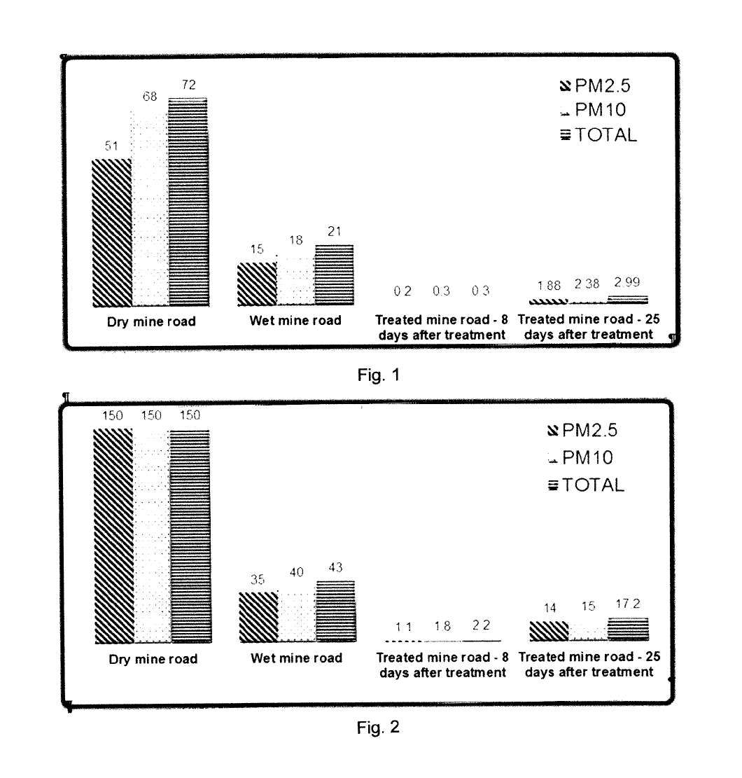

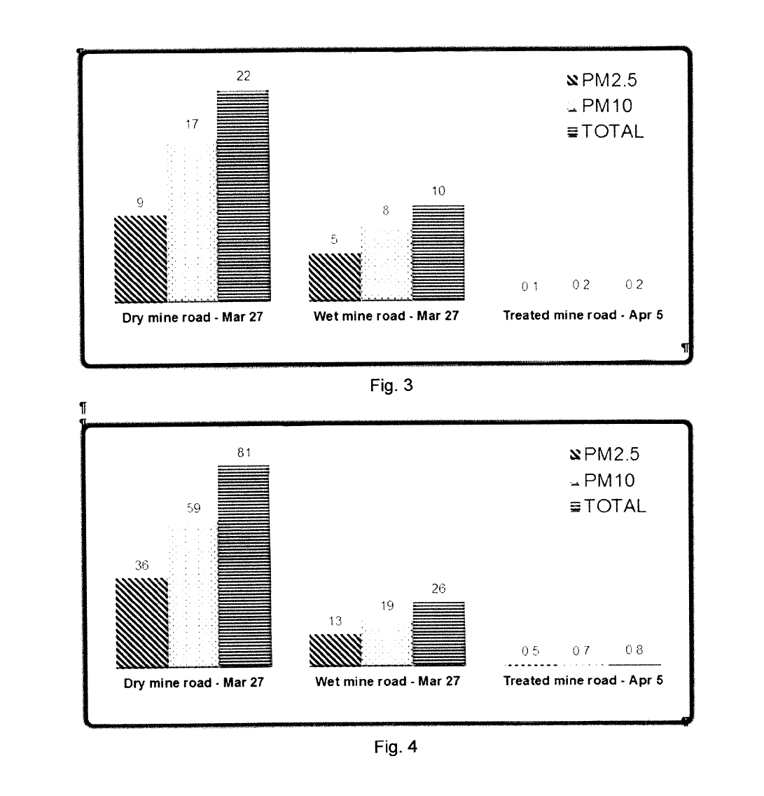

The most important measurements can be summarized using the average (FIG. 1) and maximum concentrations (FIG. 2) of dust measured as PM_{2.5}, PM₁₀ and Total PM for each road monitored using mobile monitoring, prior to the application of the product, one week after and one month after.

It is interesting to note that mobile monitoring of dry roads (first group of data on FIG. 2) indicated maximum concentrations of dust above the limit of the monitor (>150 mg/m³). For the other measurements, similar trends were observed for all the size fractions, for both maximum and average concentrations and for both the static and mobile monitoring.

Results clearly indicate that although total dust emission is reduced, for a very short period of time, by about 60% by the application of water on the road (averages: 21 mg/m³ rather than 72 mg/m³), the application of the dust suppressant is significantly more efficient, with immediate and expected long lasting dust suppression. In fact, a week after application, average concentrations of 0.3, 0.3 and 0.2 mg/m³ were calculated for the size fractions Total PM, PM₁₀ and PM₂₅, respectively. In addition, FIG. 2 indicates a low maximum concentration of Total PM of and 2.2 mg/m³ (mobile monitoring) on the treated road, a week after application. It is important to point out that the average dust concentration emitted on the road treated with the dust suppressant (0.3 mg/m³) is significantly lower than the limit of dust concentration in the workplace generally established at 15 mg/m³ for Total PM and 5 mg/m³ for respirable dusts (PM₁₀). The data reported in the last group of each FIGS. 1 and 2, indicate that an average total dust emission as low as 2.99 mg/m³ was observed a month after the application of the dust suppressant. This value is still below the limit mentioned earlier.

The road beds at mine site are well-built and prepared to undergo dust maintenance, hence the choice for the tests. The product formulation base as reported herein is well-adapted to mine site conditions. The program proved the delivery of performance and provided cost savings It also showed many advantages amongst other, i) greatly reducing dust levels (surpassing the most demanding PPM norms and standards), ii) making roads safer for heavy traffic during freezing conditions, iii) strengthening and hardening road beds, iv) avoiding use of water and harmful chemicals, v) requiring much less frequent applications compared to traditional products, and vi) allowed the team to acquire on-site knowledge.

Applying water has been the most obvious means of dust management around the world and of course at the mine site, and there are several fog and mist nozzles available for this purpose. However, the extent of dust suppression offered by this method is insufficient and the effect is short lasting, as indicated by the high dust concentration measured on the wet mine road; 26 to 43 mg/m³, which largely exceeds the usual dust emission standards in workplaces. In addition, with water shortages in some areas and the expense involved in frequent application of water on the roads, the dust suppressant solution offers an alternative of choice to control dust emission from the mine haul roads as well as from stockpiles and other point sources within the mining operation.

The application of the dust suppressant minimizes the risk to health and safety of workers, whether it is in the form of reduced visibility on haul roads or respiratory issues caused by inhaled dust, whose consequences are critical to the day-to-day mining operations. The use of non-toxic and environmentally-friendly ingredients in the formulation of the dust control products also ensures minimal environmental consequences of dust control activities by mine operators.

Reduction of dust emission by the application of the dust suppressant would address the concerns that emerged after a study into construction at mine site highlighting possible environmental damage to nearby glaciers. Controlling dust emission associated with the mining operations will minimize the dust concentration in the air, which goes on the glaciers, and mitigate the associated risk of a meltdown of glaciers.

The use of the dust suppressant will also help increase productivity and cost savings in mining operations. High levels of dust can undermine operation profitability and productivity by posing a threat to the moving parts of mining equipment required to operate the mine, which can lead to expensive repairs and downtime, or by reducing the visibility for workers which forces vehicles travelling on dusty haul roads to drive much slower.

The microbial component of the dust suppressant formulation offers additional benefits over a longer term such as bioremediation, soil regeneration, vegetation regrowth and improved “stickiness” providing stronger and better bonded roads.

### Example 2

### Field Trial II

Another field trial was carried out at the African continent, essentially as described in Example 1, with the exemption of polymer in the carrier, and an optimized treatment protocol for the variation in road bed. In this field trial the road bed had a larger clay content than in Example 1. The dust suppressant formulation was tested at a 3,750 m² area, and the driving speeds for both monitoring measurements were increased to 50 km/h.

The PM_{Total} was reduced to <0.4 ppm, corresponding to a reduction of PM of 88% and 63% respectively for the dry zone and the wet zone. The dry zone was an area of the road where nothing had been applied during the testing period, whereas the wet zone was an area sprayed with water twice a day during the testing period.

### Example 3

### Spore Formulation

The experiment was conducted to demonstrate the dust suppressing effect of bacterial spores delivered to soil in a liquid carrier.

### Materials

Sieved Slotsgrus® (Stenrand Grusgrav, Denmark), particle size ≦1 mm (autoclaved and dried); 0.45 μm Minisart® HighFlow syringe filter with Polyethersulfone (PES) membrane filter—28 mm filter, order no. 16537.

### Carrier Formulation

40% Glycerol
1% lignosulfonic acid sodium salt
Mineral salts (g/L)

2.0 NaNO₃

0.1 KCl

0.5 KH₂PO₄

1.0 K₂HPO₄

0.01 CaCl₂

0.5 MgSO₄, 7H₂O
Trace metals (mg/L)

2.75 CaCl₂, 2H₂O

6.75 FeCl₃, 6H₂O

0.50 MnCl₂, 4H₂O

0.85 ZnCl₂

0.22 CuSO₄, 5H₂O

0.55 CoCl₂, 6H₂O

0.30 (NH₄)₆Mo₇O₂₄, 4H₂O

0.34 Na₂B₄O₇, 10H₂O
Milli-Q water

### Bacterial Spores

Bacillus subtilis
Bacillus licheniformis
Bacillus amyloliquefaciens
Bacillus megaterium
The final total inoculum concentration was approx. 7×10⁷ CFU/mL.
50 mL conical centrifuge tubes

### Methods

The experiments were conducted as described below:
1. Four 1.5 mm holes were drilled in each of the 50 mL centrifuge tubes. The holes were positioned at the 20 mL line and distributed evenly around the tube.
2. 15.0 g of soil was weighed out into each tube.
3. To each of the tubes 660 μL of either full strength or diluted carrier±the spore formulation was added (according to the setup in Table 4).
4. The tubes were capped of and mixed by vigorous shaking for 5 sec.
5. Tubes were incubated w/o lids for 1 week at 30° C.
6. After incubation pre-weighed filters were fixed on top with Parafilm®. The outlet of the filter was connected to the vacuum line. The vacuum was turned up to max, and the tube was exposed to a physical treatment by vortexing for 60 sec at 3,000 rpm.
7. After the dust challenge, the amount of dust captured in the filter was monitored by weighing.

TABLE 4
Tubes 1-6 (block a) and 13-20 (block c) included full
strength carrier. Tubes 7-12 included 40% strength carrier
(diluted in water)(block b). The block column indicates
the sorting needed for statistical modeling.

| Tube | Treatment with Spores | Block |
|---|---|---|
| 1 - 7 - 13 | Yes | a - b - c |
| 2 - 8 - 14 | No | a - b - c |
| 3 - 9 - 15 | Yes | a - b - c |
| 4 - 10 - 16 | No | a - b - c |
| 5 - 11 - 17 | No | a - b - c |
| 6 - 12 - 18 | Yes | a - b - c |
| 19 | No | c |
| 20 | Yes | c |

### Results

A total of 20 samples were evaluated in three separate runs (block a, b, c in Table 4). The output was analyzed using SAS JMP® having the data sorted in blocks according to run, and fitting a model with standard least squares to reach comparable dust levels between runs. The results are shown in Table 5.

TABLE 5
Amount of dust (mg) captured in a 0.45 μm filter, in the presence
or absence of spores. Each value is an average of 10 samples.

| Treatment with spores | Average amount of dust |
|---|---|
| Yes | 424 mg |
| No | 506 mg |

### Conclusion

The data show that microbial spores can significantly increase the dust-suppressing capacity of a liquid carrier.

### Example 4

### Non-Glycerol Carrier Formulation

The experiment was conducted to evaluate the dust suppressing effect of bacterial spores delivered to soil in a different liquid carrier supporting growth of the bacterial spores.

### Materials

Sieved Slotsgrus® (Stenrand Grusgrav, Denmark), particle size ≦1 mm (autoclaved and dried)

### Carrier Formulation

50% v/v TY broth
10% v/v TY broth

### Bacterial Spores

Bacillus subtilis
Bacillus licheniformis
Bacillus amyloliquefaciens
Bacillus megaterium

The final total inoculum concentration was approx. 7×10⁷ CFU/mL.

50 mL conical centrifuge tubes

### Methods

The experiments were conducted as described below:
1. Four 1.5 mm holes were drilled in each of the 50 mL centrifuge tubes. The holes were positioned at the 20 mL line and distributed evenly around the tube.
2. 15.0 g of soil was weighed out into each tube.
3. To each of the tubes 660 μL of either 50% v/v or 10% v/v carrier±the spore formulation was added.
4. The tubes were capped of and mixed by vigorous shaking for 5 sec.
5. Tubes were incubated for 1 week at 30° C. with two repeated addition of fresh TY-broth in the same concentration as the initial addition.
6. After incubation an outlet was coupled to a handheld particle measurer (Lighthouse 3016), and dust was monitored as particles between 2.0 μm and 5.0 μm reported as number of particles/m³. Each measurement sampled ˜1 L of air (equivalent to 21 seconds of collecting air). The tubes were exposed to physical treatment by vortexing at 300 rpm.
7. The experimental setup is shown in Table 6.

TABLE 6
Tubes were numbered 1-12.

| Tube | Treatment with Spores | TY-broth (% v/v) |
|---|---|---|
| 1 | No | 50 |
| 2 | No | 10 |
| 3 | No | 10 |
| 4 | Yes | 10 |
| 5 | Yes | 50 |
| 6 | No | 10 |
| 7 | Yes | 10 |
| 8 | Yes | 10 |
| 9 | Yes | 50 |
| 10 | Yes | 50 |
| 11 | No | 50 |
| 12 | No | 50 |

### Results

A total of 12 samples were evaluated in a single run. The output was analyzed using SAS JMP® and fitting a model with standard least squares. The results are shown in Table 7.

TABLE 7
Average number of dust-particles (between 2.0 and 5.0 μm) per
m³ of air released after vortexing at 300 rpm, and collecting ~1 L
of air for quantification - with or without treatment with spores.

| Treatment with spores | Average number of particles per m³ |
|---|---|
| Yes | 39351 |
| No | 66930 |

### Conclusion

The data show that treatment with microbial spores in a non-glycerol carrier can significantly reduce the number of particles released from a soil sample, which has been exposed to a physical treatment (vortexing).

### Example 5

### Soil Dust Suppression

The experiment was conducted to demonstrate the dust suppressing effect of bacterial spores delivered to soil in a liquid carrier.

### Materials

Ambient humidity soil

Dust suppressant formulation

The dust suppression composition of Example 1 was used.

A control with only crude glycerol (47%) was employed as control without microbial spores—denoted without microbial spores

### Methods

The experiments were conducted as described below:
1. 1.1 mL of the two different formulations was mixed, using a spatula, with 25 g of soil. Three replicates were prepared for each time-point resulting in a total of 12 samples.
2. The samples were covered with punctured aluminum foil and incubated at 32° C. for either one or two weeks.
3. Dust emission was evaluated by dropping the sample through a 5 ft tall, 6″ wide tube having a Dust Track Monitor mounted two thirds down.
4. Results are monitored as maximum ppm concentration.

### Results

A total of 12 samples were evaluated over the course of the two weeks, in parallel with samples of 25 g of soil without the crude glycerol and the spores (negative control). The output was analyzed using SAS JMP® and fitting a model with standard least squares. The results are shown in Table 8.

TABLE 8
Average concentration of dust-particles (ppm) released after
dropping soil sample through 5 ft tall tube - with or without
treatment with spores. The data is sorted by weeks of incubation.
Maximum reading on the equipment is 150 ppm.

| Incubation time | Treatment with spores | Average particles (ppm) |
|---|---|---|
| 1 week | Negative control | >150 |
| | Yes | 13 |
| | No | 81 |
| 2 weeks | Negative control | >150 |
| | Yes | 77 |
| | No | 139 |

### Conclusion

The data show that treatment with microbial spores can significantly reduce the number of particles released from a soil sample, which has been exposed to a physical treatment (dropping).

### Example 6

### Coal Dust Suppression

The experiment was conducted to demonstrate the dust suppressing effect on materials other than silica based, as has been shown in the other examples. This example is to show the dust suppressing effect when applying microbial spores in a liquid carrier to coal particles.

### Materials

Ambient humidity coal dust

### Dust Suppressant Formulation

The dust suppression composition of Example 1 was used.

A control with only crude glycerol (47%) was employed as control without microbial spores—denoted without microbial spores

### Methods

The experiments were conducted as described below:
1. 1.1 mL of the two different formulations was mixed, using a spatula, with 25 g of coal dust. Three replicates were prepared for each treatment resulting in a total of 6 samples.
2. The samples were covered with punctured aluminum foil and incubated at 32° C. for one week.
3. Dust emission was evaluated by dropping the sample through a 5 ft tall, 6″ wide tube having a Dust Track Monitor mounted two thirds down.
4. Results are monitored as maximum ppm concentration.

### Results

A total of 6 samples were evaluated. The output was analyzed using SAS JMP® and fitting a model with standard least squares. The results are shown in Table 9.

TABLE 9
Average concentration of dust-particles (ppm) released after dropping
soil sample through 5 ft tall tube - with or without treatment
with spores. Maximum reading on the equipment is 150 ppm.

| Treatment with spores | Average particles (ppm) |
|---|---|
| Yes | 97 |
| No | >150 |

Conclusion

The data show that treatment with microbial spores can significantly reduce the number of particles released from a coal dust sample, which has been exposed to a physical treatment (dropping).

### Example 7

### Effect of Growth State of Bacteria

The experiment was conducted to evaluate the dust suppressing effect of bacteria in relation to its growth state. This example compares spores to exponential growing cells. The effect was evaluated in TY-broth as carrier to ensure continued exponential growth of the bacterial cells.

### Materials

Sieved Slotsgrus® (Stenrand Grusgrav, Denmark), particle size ≦1 mm

### Carrier Formulation

TY: 10% v/v TY broth

### Bacterial Composition

Bacillus subtilis

Bacillus licheniformis

Bacillus amyloliquefaciens

Bacillus megaterium

The final total inoculum concentration of spores and exponentially growing cells was approx. 7×10⁷ CFU/mL.

50 mL conical centrifuge tubes

### Methods

The experiments were conducted as described below:
1. Four 1.5 mm holes were drilled in each of the 50 mL centrifuge tubes. The holes were positioned at the 20 mL line and distributed evenly around the tube.
2. 15.0 g of soil was weighed out into each tube.
3. A bacterial culture was prepared and grown to reach exponential growth phase in TY broth, upon which it was centrifuged and supernatant was discarded.
4. To each of the tubes 660 μL of carrier ±bacterial formulation was added.
5. The tubes were capped of and mixed by vigorous shaking for 5 sec.
6. Tubes were incubated for 1 week at 30° C. with two repeated addition of fresh carrier without bacteria.
7. After incubation an outlet was coupled to a handheld particle measurer (Lighthouse 3016), and dust was monitored as particles between 2.0 μm and 5.0 μm reported as number of particles/m³. Each measurement sampled ˜1 L of air (equivalent to 21 seconds of collecting air). The tubes were exposed to physical treatment by vortexing at 300 rpm.
8. The experimental plan is shown in Table 10.

TABLE 10
Tubes were numbered 1-9.

| Tube | Bacteria (and growth state) |
|---|---|
| 1 | Spore |
| 2 | No |
| 3 | No |
| 4 | Spore |
| 5 | Exponential |
| 6 | Exponential |
| 7 | Exponential |
| 8 | Spore |
| 9 | No |

### Results

A total of 9 samples were evaluated in a single run. The results are shown in Table 11.

TABLE 11
Average number of dust-particles (between 2.0 and 5.0 μm) per
m³ of air released after vortexing at 300 rpm, and collecting ~1
L of air for quantification - with or without treatment with
bacteria in different growth states and TY-broth as carrier.

| Bacteria (and growth state) | Average number of particles per m³ |
|---|---|
| No bacteria | 71638 |
| Exponential growth | 68724 |
| Spore | 58073 |

### Conclusion

The data show that treatment with microbial spores can significantly reduce the number of particles released from a soil sample, which has been exposed to a physical treatment (vortexing), and that the effect is reduced if bacteria in inoculum are not of spore origin.

It will be understood that the Specification and Examples are illustrative of the present embodiments and that other embodiments within the spirit and scope of the claimed embodiments will suggest themselves to those skilled in the art. Although this invention has been described in connection with specific forms and embodiments thereof, it would be appreciated that various modifications other than those discussed above may be resorted to without departing from the spirit or scope of the invention as defined in the appended claims. For example, equivalents may be substituted for those specifically described, and in certain cases, particular applications of steps may be reversed or interposed all without departing from the spirit or scope for the invention as described in the appended claims.

1-30. (canceled)

31. A dust suppressant liquid composition comprising a liquid carrier and one or more dust supressing spore forming bacterial strains.

32. The composition of claim 31, wherein the one or more dust suppressing spore forming bacterial strains are selected from the genera Acetonema, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridiisalibacter, Cohnella, Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planifilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, and Vulcanobacillus.

33. The composition of claim 31, wherein the one or more dust suppressing spore forming bacterial strains comprises one or more strains of Bacillus.

34. The composition of claim 33, wherein the one or more strains of Bacillus comprise one or more bacterial strains of Bacillus subtilis, Bacillus licheniformis, Bacillus amyloliquefaciens, or Bacillus megaterium.

35. The composition of claim 31, wherein the one or more dust suppressing spore forming bacterial strains are selected from the group consisting of:
Bacillus subtilis;
Bacillus licheniformis;
Bacillus amyloliquefaciens;
Bacillus megaterium;
Bacillus subtilis, and Bacillus licheniformis;
Bacillus subtilis, and Bacillus amyloliquefaciens;
Bacillus subtilis, and Bacillus megaterium;
Bacillus licheniformis, and Bacillus amyloliquefaciens;
Bacillus licheniformis, and Bacillus megaterium;
Bacillus amyloliquefaciens, and Bacillus megaterium;
Bacillus subtilis, Bacillus licheniformis, and Bacillus amyloliquefaciens;
Bacillus subtilis, Bacillus licheniformis, and Bacillus megaterium;
Bacillus licheniformis, Bacillus amyloliquefaciens, and Bacillus megaterium; and
Bacillus subtilis, Bacillus licheniformis, Bacillus amyloliquefaciens, and Bacillus megaterium.

36. The composition of claim 31, wherein the liquid carrier comprises water.

37. The composition of claim 31, wherein the liquid carrier is an aqueous solution.

38. The composition of claim 37, wherein the aqueous solution comprises glycerine.

39. The composition of claim 38 further comprising a natural polymer.

40. A method for suppressing dust, comprising applying the dust suppressant composition as defined in claim 31 to a substrate.

41. The method of claim 40, wherein the substrate is a road.

42. The method of claim 41, wherein the road is a mining road.

43. A method for reducing dust, comprising applying the dust suppressant composition as defined in claim 31 to a substrate.

44. The method of claim 43, wherein said substrate is a road.

45. The method of claim 43, wherein said composition is applied at a rate of between 5,000 to 10,000 gallons per km.

46. The method of claim 44, wherein said composition is allowed to sit on said road for at least 24 hours prior to traffic being allowed to travel on the road.

47. A method for reducing or preventing formation of dust from a soil surface, comprising applying the composition of claim 31 to the soil surface.

48. The method claim 47, wherein the soil surface is a road surface.

49. The method claim 47, which retains reduced dust formation after a month from applying the composition to the soil surface.
